(19) United States
(12) Patent Application Publication
(10) Pub. No.: US 2023/0123697 A1
(43) Pub. Date: Apr. 20, 2023

(54) THERAPEUTIC PATCH FOR GASTROINTESTINAL TRACT AND METHOD OF MANUFACTURING SAME

(71) Applicants: DAEGU GYEONGBUK INSTITUTE OF SCIENCE AND TECHNOLOGY [DGIST], Daegu (KR); INDUSTRY-ACADEMIC COOPERATION FOUNDATION, YONSEI UNIVERSITY, Seoul (KR)

(72) Inventors: Suk Ho PARK, Gwangju (KR); Ji-Hun LEE, Busan (KR); Seung Min BANG, Seoul (KR)

(21) Appl. No.: 17/879,926

(22) Filed: Aug. 3, 2022

(30) Foreign Application Priority Data
Oct. 20, 2021 (KR) 10-2021-0140276

(51) Int. Cl. A61K 9/00; A61K 41/00; A61K 9/51

(52) U.S. Cl. CPC A61K 9/0065; A61K 41/0052; A61K 9/5161; A61K 9/0009; A61K 9/5192

(57) ABSTRACT

Disclosed is a therapeutic patch for the gastrointestinal tract including a mucoadhesive material along with magnetic nanoparticles and a drug to be delivered to a living body, which are supported on the mucoadhesive material. A method of manufacturing the therapeutic patch for the gastrointestinal tract is also provided and includes preparing a mucoadhesive polymer, preparing a catechol precursor, mixing the mucoadhesive polymer and the catechol precursor, freeze-drying a mixture in the mixing step, mixing a powder resulting from the freeze-drying step with magnetic nanoparticles and a drug, and subjecting the resultant mixture to molding using a mold and then freeze-drying.

### BACKGROUND OF THE INVENTION

### Field of the Invention

Various embodiments of the present invention relate to a therapeutic patch for the gastrointestinal tract and a method of manufacturing the same. More particularly, the present invention relates to a therapeutic patch for the gastrointestinal tract, which has excellent mucosal adhesion to the gastrointestinal tract and enables drug release and hyperthermia, and a method of manufacturing the same.

### Description of the Related Art

With the proportionate increase in society of the aging population, which is dependent on social support, effective and economical treatment for various kinds of diseases is required. Accordingly, effective medical technology capable of appropriately administering a necessary amount of a therapeutic agent to an affected area depending on the patient's condition using a drug delivery system (DDS) technique is being developed. The initial drug delivery technique was developed focusing on therapeutic materials, but since then, research is ongoing not only into therapeutic drugs but also into drug delivery systems, that is, into methods for efficient delivery thereof. In particular, recently, a targeted drug delivery system capable of realizing increased drug delivery efficiency is being developed.

In the related art, various types of drugs (lipid structures, nanoparticles, liposomes, osmotic agents, etc.) and drug carriers are under study, and these drugs and drug carriers are delivered into the human body using vascular (venous) and intramuscular injection, oral drugs, patches, etc. However, these methods are usually conducted in a manner in which a drug is delivered by being absorbed into blood vessels. Here, a patch has the ability to deliver the drug locally to the skin. However, existing drug delivery methods do not deliver drugs directly to diseased sites in the digestive organ, but deliver drugs through blood vessels, so they have limitations on drug delivery efficiency, and various drug side effects may occur due thereto.

### SUMMARY OF THE INVENTION

The present invention has been made keeping in mind the problems encountered in the related art and is intended to provide a therapeutic patch for the gastrointestinal tract, which has excellent mucosal adhesion to the gastrointestinal tract and is sufficiently attached to a target lesion site to thus actively deliver a drug and enable hyperthermia, and a method of manufacturing the same.

A therapeutic patch for the gastrointestinal tract according to various embodiments of the present invention may include a mucoadhesive material along with magnetic nanoparticles and a drug to be delivered to a living body, which are supported on the mucoadhesive material.

A method of manufacturing a therapeutic patch for the gastrointestinal tract according to various embodiments of the present invention may include preparing a mucoadhesive polymer, preparing a catechol precursor, mixing the mucoadhesive polymer and the catechol precursor, freeze-drying a mixture in the mixing step, mixing a powder resulting from the freeze-drying step, magnetic nanoparticles, and a drug, and subjecting the resultant mixture to molding using a mold and then freeze-drying.

### BRIEF DESCRIPTION OF THE DRAWINGS

The above and other objects, features, and other advantages of the present invention will be more clearly understood from the following detailed description taken in conjunction with the accompanying drawings, in which:

FIG. 1 is a schematic view showing a therapeutic patch for the gastrointestinal tract according to various embodiments of the present invention;

FIG. 2 is a view for explaining a process of treating the gastrointestinal tract using the therapeutic patch for the gastrointestinal tract according to the present invention;

FIG. 3 is a flowchart showing a process of manufacturing a therapeutic patch for the gastrointestinal tract according to various embodiments of the present invention;

FIG. 4 is a photograph showing the therapeutic patch for the gastrointestinal tract manufactured according to an embodiment of the present invention; and

FIG. 5 is a graph showing the results of a test of adhesion of the therapeutic patch for the gastrointestinal tract manufactured according to an embodiment of the present invention.

### DETAILED DESCRIPTION OF THE INVENTION

Hereinafter, various embodiments of the present document will be described with reference to the accompanying drawings. The embodiments and terms used herein are not intended to limit the technology described in this document to specific exemplary embodiments, but they should be understood to cover various modifications, equivalents, and/or substitutions of the embodiments.

With reference to the accompanying drawings, a detailed description will be given of embodiments of the present invention below.

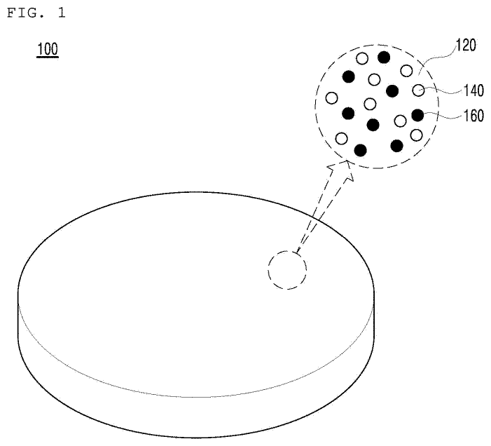

As shown in FIG. 1, a therapeutic patch 100 for the gastrointestinal tract according to various embodiments of the present invention includes a mucoadhesive material 120, magnetic nanoparticles 140, and a drug 160.

The mucoadhesive material 120 serves as a base material in the therapeutic patch 100 for the gastrointestinal tract, and magnetic nanoparticles 140 and a drug 160 may be supported thereon.

The mucoadhesive material 120 may include a mucoadhesive polymer having a catechol structure introduced thereto. The mucoadhesive material 120 may be a mucoadhesive-polymer/catechol conjugate. Specifically, the mucoadhesive polymer may be at least one selected from the group consisting of chitosan, alginic acid, guar gum, xanthan gum, pectin, galactomannan, glucomannan, hyaluronic acid, glycosaminoglycan, gelatin, polyethylene glycol, polyethylene oxide, polyacrylic acid, polymethacrylic acid, polyvinyl pyrrolidone, polyvinyl amine, and derivatives thereof. The mucoadhesive material 120 may be formed by introducing the catechol structure to the mucoadhesive polymer.

For example, the mucoadhesive material 120 may be prepared by introducing a crosslinking functional group into the mucoadhesive polymer, introducing a crosslinking functional group into catechol, and performing free radical polymerization. For example, the crosslinking functional group may be any one selected from the group consisting of acrylic double bonds, such as methacrylate, ethacrylate, ethylmaleato, ethylfumarato, N-maleimido, vinyloxy, alkylvinyloxy, vinylmaleato, and vinylfumarato groups.

Catechol is a functional group of the mussel adhesive protein. When introducing the catechol structure, hydrocaffeic acid (HCA), which is a catechol precursor, may be used. Here, the ratio of the mucoadhesive polymer and HCA that are introduced may be 1:1-1.2. Also, the degree of substitution of catechol in the mucoadhesive-polymer/catechol conjugate may be 5% to 20%. Through such a composition, it is possible to realize mucosal adhesion and a hemostatic function in the gastrointestinal tract.

The magnetic nanoparticles 140 may be supported on the mucoadhesive material 120. The magnetic nanoparticles 140 may be iron oxide magnetic nanoparticles. For example, the magnetic nanoparticles 140 may include at least one of Fe₂O₃ and Fe₃O₄. The magnetic nanoparticles 140 may be moved in position by an external magnetic field, and may generate heat under an external stimulus such as an alternating magnetic field (AMF) or near-infrared (NIR).

The magnetic nanoparticles 140 may have a diameter of 1 nm to 500 nm. The magnetic nanoparticles 140 may be coated with chitosan. The chitosan may be chemically bound to the mucoadhesive material 120. For example, —NH₂ of chitosan and —COOH of catechol may be bound through carbodiimide chemistry. Thereby, the drug 160 may be preferentially released after the therapeutic patch 100 for the gastrointestinal tract is delivered to the target lesion site. Moreover, the magnetic nanoparticles 140 may generate heat under an external stimulus, so hyperthermia of the target lesion site may be effectively performed.

Also, when manufacturing the therapeutic patch 100 for the gastrointestinal tract, the magnetic nanoparticles 140 are mixed with deionized (DI) water. Here, the magnetic nanoparticles 140 may be included in an amount of 5 mg/ml to 30 mg/ml based on the amount of DI water that is added. If the amount of the magnetic nanoparticles 140 is less than 5 mg/ml, the heating function may deteriorated, whereas if the amount of the magnetic nanoparticles 140 is greater than 30 mg/ml, they may not be dissolved in DI water, and patch molding may become problematic.

The drug 160 may be a drug that is delivered to a target lesion site and has various therapeutic effects. The drug 160 may be supported on the mucoadhesive material 120, and may then be actively released due to the elevated temperature after the therapeutic patch 100 for the gastrointestinal tract reaches the target lesion site. The diameter of the drug 160 may be smaller than the diameter of the magnetic nanoparticles 140. Thereby, the release of the drug 160 may be effectively induced.

When manufacturing the therapeutic patch 100 for the gastrointestinal tract, the drug 160 is mixed with DI water. Here, the drug 160 may be added in an amount of 0.5 mg/ml to 3 mg/ml based on the amount of DI water that is added. If the amount of the drug 160 is less than 0.5 mg/ml, drug treatment efficacy may deteriorated, whereas if the amount of the drug 160 is greater than 3 mg/ml, the drug may not be dissolved in DI water when manufacturing the patch. However, embodiments of the present invention are not limited thereto, and the amount of the drug that is added may vary depending on the type of drug 160.

The therapeutic patch 100 for the gastrointestinal tract may be in the form of a circular disk having a diameter of 4 mm to 12 mm. The therapeutic patch 100 for the gastrointestinal tract has an appropriate area, so it may be attached to the site of bleeding in the gastrointestinal tract, thus effectively forming a hemostatic film.

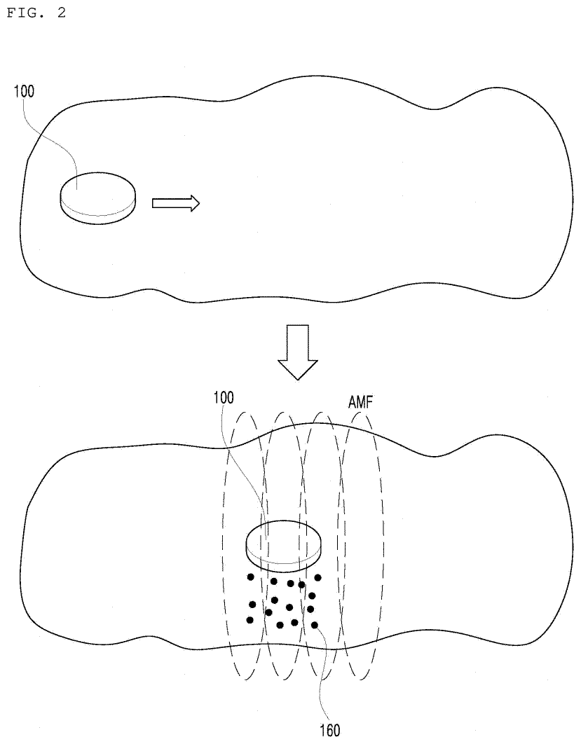

With reference to FIG. 2, a method of treating the gastrointestinal tract using the therapeutic patch 100 for the gastrointestinal tract according to the present invention is described.

The therapeutic patch 100 for the gastrointestinal tract may be delivered to a target lesion site in the gastrointestinal tract (small intestine, large intestine, stomach, etc.) through wired endoscopy or capsule endoscopy.

The therapeutic patch 100 for the gastrointestinal tract may be effectively attached to the target lesion site by the mucoadhesive material 120. Next, an external stimulus such as an alternating magnetic field (AMF) or near-infrared (NIR) is applied so that the magnetic nanoparticles 140 included in the therapeutic patch 100 for the gastrointestinal tract generate heat, thereby elevating the temperature of the therapeutic patch 100 for the gastrointestinal tract. Due to the elevation in the temperature of the therapeutic patch 100 for the gastrointestinal tract, the drug 160 may be actively released, so chemical treatment may be carried out. Moreover, due to the elevation in the temperature of the therapeutic patch 100 for the gastrointestinal tract, hyperthermia of the lesion site may be concurrently performed. Also, when the lesion site is a bleeding site, the therapeutic patch 100 for the gastrointestinal tract may provide a hemostatic effect by forming a hemostatic film.

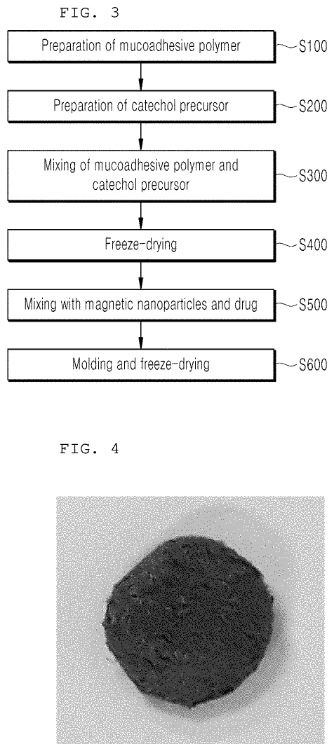

With reference to FIG. 3, a method of manufacturing the therapeutic patch for the gastrointestinal tract according to various embodiments of the present invention is specified below.

The method of manufacturing the therapeutic patch for the gastrointestinal tract according to various embodiments of the present invention may include preparing a mucoadhesive polymer (S100), preparing a catechol precursor (S200), mixing the mucoadhesive polymer and the catechol precursor (S300), freeze-drying a mixture in the mixing step (S400), mixing a powder resulting from the freeze-drying step, magnetic nanoparticles, and a drug (S500), and subjecting the resultant mixture to molding using a mold and then freeze-drying (S600).

First, in the step of preparing the mucoadhesive polymer (S100), at least one selected from the group consisting of chitosan, alginic acid, guar gum, xanthan gum, pectin, galactomannan, glucomannan, hyaluronic acid, glycosaminoglycan, gelatin, polyethylene glycol, polyethylene oxide, polyacrylic acid, polymethacrylic acid, polyvinyl pyrrolidone, polyvinyl amine, and derivatives thereof may be prepared. A mucoadhesive polymer solution at a pH of 5.5 may be prepared by mixing the mucoadhesive polymer with hydrochloric acid (HCl) and deionized distilled water.

Next, in the step of preparing the catechol precursor (S200), hydrocaffeic acid (HCA), serving as the catechol precursor, and EDC (1-ethyl-3-(3-dimethylaminopropyl)carbodiimide hydrochloride) may be mixed with ethanol.

In the step of mixing the mucoadhesive polymer and the catechol precursor (S300), the mucoadhesive polymer solution and the catechol precursor, which are prepared as described above, are mixed, and are then allowed to react at a pH adjusted to 5.0. Thereafter, dialysis may be performed to remove unreacted materials. Here, the ratio of the mucoadhesive polymer and HCA that are introduced may be 1:1-1.2.

In the freeze-drying step (S400), the mixed solution of the mucoadhesive polymer and the catechol precursor may be freeze-dried. The freeze-drying step (S400) may be performed at −70° C. to −90° C. Within the above temperature range, it is possible to reduce the pore size of the patch to be manufactured and enhance adhesion thereof.

In the step of mixing the magnetic nanoparticles and the drug (S500), the powder resulting from the freeze-drying step (S400), the magnetic nanoparticles, and the drug may be mixed with water. Here, the magnetic nanoparticles may be magnetic nanoparticles coated with chitosan. —NH₂ of chitosan and —COOH of catechol may be bound through carbodiimide chemistry.

Next, in the step of molding and then freeze-drying (S600), the mixture obtained in S500 may be molded using a mold in the form of a patch and then freeze-dried.

A better understanding of the present invention may be obtained through the following example and test example. However, the following example and test example are merely set forth to illustrate the present invention, and are not construed as limiting the present invention.

### EXAMPLE

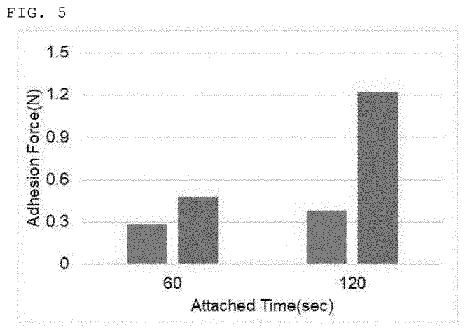

A chitosan solution was prepared by mixing 1 g of chitosan, 5 ml of HCl (hydrochloric acid), and 44.5 ml of DDW (deionized distilled water). The chitosan solution was mixed to homogeneity, after which the pH thereof was adjusted to 5.5. 1.18 g of hydrocaffeic acid (HCA), serving as a catechol precursor, and EDC (1-ethyl-3-(3-dimethylaminopropyl)carbodiimide hydrochloride) (1.24 g, 8.0 mmol) were mixed respectively with 5 ml and 20 ml of ethanol, after which the resulting mixture was mixed with the chitosan solution. The pH of the mixed solution was adjusted to 5.0, after which the reaction was allowed to proceed for 12 hours. Dialysis was performed in a NaCl solution (10 mM, MWCO 3500) at a pH of 3.5 for two days. Thereafter, dialysis was performed in DI water for 4 hours. Thereafter, the solution was frozen at −80° C. and then freeze-dried. A drug-supported chitosan/catechol solution was prepared by mixing the freeze-dried mucoadhesive material, magnetic nanoparticles, and a drug with water. The drug-supported chitosan/catechol solution was placed in a patch-shaped mold, frozen at −80° C., and freeze-dried, thereby obtaining a therapeutic patch for the gastrointestinal tract as shown in FIG. 4.

### Test Example—Adhesion Test Depending on Freezing Temperature

Variation in adhesion depending on the freezing temperature in the freezing step before freeze-drying was measured. A patch was frozen in each of a refrigerator at −4° C. and a deep freezer at −80° C., and adhesion thereof to the small intestine of a pig was measured using a load cell. Each patch was held in place for 60 seconds or 120 seconds, and the results of testing of adhesion thereof are shown in Table 1 below.

TABLE 1

| | | Time (s) | |
|---|---|---|---|
| | | 60 | 120 |
| Adhesion (N) | Refrigerator | 0.28 | 0.3808 |
| | Deep freezer | 0.4802 | 1.22 |

As shown in Table 1, it was found that the adhesion of the patch frozen in the deep freezer at −80° C. was much greater than that of the patch frozen in the refrigerator at −4° C. Therefore, it was confirmed that the freezing temperature is also an important factor in the adhesion of the patch.

As is apparent from the above description, a therapeutic patch for the gastrointestinal tract according to various embodiments of the present invention has excellent mucosal adhesion in the gastrointestinal tract, thereby enhancing the drug treatment effect. The therapeutic patch for the gastrointestinal tract according to the present invention is effective at performing chemical treatment by actively releasing a drug to a target lesion site using heating through magnetic nanoparticles. Moreover, due to the increase in the temperature of the therapeutic patch for the gastrointestinal tract, hyperthermia can be performed concurrently. The therapeutic patch for the gastrointestinal tract according to the present invention has an appropriate area, so it can be attached to the site of bleeding in the gastrointestinal tract to thus effectively form a hemostatic film, thereby performing an excellent hemostatic function.

The features, structures, effects, and the like described in the embodiments are included in at least one embodiment of the present invention, but are not necessarily limited to one embodiment. Moreover, the features, structures, effects, and the like illustrated in each embodiment may be combined or modified for other embodiments by those of ordinary skill in the art to which the embodiments belong. Accordingly, content related to such combinations and modifications should be interpreted as being included in the scope of the present invention.

In addition, although the embodiments have been described above, these are merely exemplary and do not limit the present invention, and those of ordinary skill in the art to which the present invention belongs will appreciate that various modifications and applications not exemplified above are possible without departing from the essential characteristics of the present embodiment. For example, each component specifically shown in the embodiments may be implemented in a modified form. Also, differences related to these modifications and applications should be construed as being included in the scope of the present invention defined in the appended

claims.

What is claimed is:

1. A therapeutic patch for a gastrointestinal tract, comprising:
a mucoadhesive material; and
magnetic nanoparticles and a drug to be delivered to a living body, which are supported on the mucoadhesive material.

2. The therapeutic patch of claim 1, wherein the mucoadhesive material comprises a mucoadhesive polymer having a catechol structure introduced thereto.

3. The therapeutic patch of claim 2, wherein the mucoadhesive polymer is at least one selected from the group consisting of chitosan, alginic acid, guar gum, xanthan gum, pectin, galactomannan, glucomannan, hyaluronic acid, glycosaminoglycan, gelatin, polyethylene glycol, polyethylene oxide, polyacrylic acid, polymethacrylic acid, polyvinyl pyrrolidone, polyvinyl amine, and derivatives thereof.

4. The therapeutic patch of claim 1, wherein a degree of substitution of catechol in the mucoadhesive material is 5% to 20%.

5. The therapeutic patch of claim 1, wherein the magnetic nanoparticles are iron oxide magnetic nanoparticles.

6. The therapeutic patch of claim 1, wherein the magnetic nanoparticles are coated with chitosan.

7. The therapeutic patch of claim 2, wherein the magnetic nanoparticles are coated with chitosan, and —NH₂ of the chitosan and —COOH of the catechol are bound through carbodiimide chemical bonding.

8. The therapeutic patch of claim 1, wherein the therapeutic patch for the gastrointestinal tract is actively delivered to a lesion site through wired endoscopy or capsule endoscopy.

9. A method of manufacturing a therapeutic patch for a gastrointestinal tract, comprising:
preparing a mucoadhesive polymer;
preparing a catechol precursor;
mixing the mucoadhesive polymer and the catechol precursor;
freeze-drying a mixture in the mixing the mucoadhesive polymer and the catechol precursor;
mixing a powder resulting from the freeze-drying the mixture with magnetic nanoparticles and a drug; and
subjecting a resultant mixture to molding using a mold and then freeze-drying.

10. The method of claim 9, wherein the catechol precursor is hydrocaffeic acid (HCA).

11. The method of claim 9, wherein the mucoadhesive polymer and the catechol precursor are mixed at a ratio of 1:1-1.2.

12. The method of claim 9, wherein the freeze-drying is performed at −70° C. to −90° C.
